(19) United States
(12) Patent Application Publication
(10) Pub. No.: US 2006/0137684 A1
(43) Pub. Date: Jun. 29, 2006

(54) Compositions and methods of treating irritation and kit therefor

(76) Inventors: Celeste Evans, North Brunswick, NJ (US); Meghan Russell, Ewing, NJ (US); Daniel Weiss, New York, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/285,811

(22) Filed: Nov. 23, 2005

Related U.S. Application Data
(60) Provisional application No. 60/632,151, filed on Dec. 1, 2004.

(51) Int. Cl. A61K 31/7048; A61K 31/7034; A61K 31/496; A61K 31/551; A61K 31/4184; A61K 31/4178; A61K 31/522; A61K 31/19

(52) U.S. Cl. 128/200.23; 514/28; 514/35; 514/37; 514/39; 514/254.07; 514/396; 514/397; 514/220; 514/263.32; 514/560; 514/575

(57) ABSTRACT

This invention relates to methods, compositions, kits and treatment regimens for applying active ingredients to the groin area of an individual to treat the symptoms of a vaginal or vulvar condition using a spray applicator.

### BACKGROUND OF THE INVENTION

The groin area of both men and women represents an area that can easily become significantly irritated for various reasons.

Men, especially those involved in sporting activities, frequently suffer from fungal infections, which leads to itching and rashes.

Women also suffer from conditions that result in itching, pain and general discomfort in the vaginal and perineum area. Many things can lead to these conditions including a change in the women's vaginal pH, infection, shaving or hair removal, allergic reaction, menopause, and actions such as childbirth (including episiotomies), douching and sexual relations. Whenever a women's vaginal pH system is disrupted, the vagina and perineum can become irritated, resulting in the above conditions. Additionally, any type of clothing that causes rubbing may also irritate the vaginal tissues. Such clothing includes undergarments and pants.

In particular, the organism Candida albicans has caused women a great deal of suffering, especially due to vaginal yeast infections. If not treated promptly and appropriately, such vaginal yeast infections can become increasingly uncomfortable and result in the above conditions. These types of infections typically have been treated by conventional treatment types: systemically, topically_(including intravaginally) or a combination of treatment types. Systemic treatments include oral administration of antifungal compositions, such as Diflucan® tablets. Topical treatments include vaginal application of creams, suppositories, soft gelatin capsules, vaginal tablets and vaginal ointments. These treatments are generally available in treatment regimens ranging from one to seven days.

In many cases, vaginal yeast infections are not restricted to the vaginal area only. In fact, the infections may extend to and involve the external vulvar and perineum area as well, causing discomfort and itching externally as well as internally. Various combination regimens have become available to treat such infections both internally and externally including creams, suppositories or ovules intended for internal insertion and creams intended for external application to the vulva to relieve itching, pain and discomfort in the vulvar area.

While women suffering the pain and discomfort of vaginal yeast infections may find relief eventually during the course of treatment with the above, known regimens, they may not perceive this relief for several days. Although the application of external vaginal creams may offer some relief during the course of treatment, this relief is not immediate or instantaneous. The infection continues to produce a lumpy, cheesy discharge that heightens the discomfort caused by the infection.

Notwithstanding, the oral treatments available for the external pain and itching of vaginal infections or conditions do not provide rapid or fast relief Topical cream treatments containing active ingredients are available, however, these are currently applied by hand and entail placing the hand on the irritated skin as well as placing cream or lotion on the hand. This can be extremely messy and is, to some women, objectionable.

Both sexes may also suffer from rectal or anal conditions such as hemorrhoids, which can result in itching and burning.

### SUMMARY OF THE INVENTION

The method of this invention relates to spraying a composition containing at least one ameliorating ingredient, which is contained in a spray bottle capable of spraying in an attitude 180° displaced from its normal attitude with respect to the ground, i.e., “inverted”, on the affected groin area.

In another embodiment, this invention relates to a method of treating a vaginal condition comprising spraying a perineum with a composition comprising at least one ameliorating agent.

In still another embodiment, this invention relates to a method of treating a groin condition comprising spraying a groin area with a composition comprising an ameliorating agent.

In yet another embodiment, this invention relates to a method of treating a vaginal condition comprising cleaning an affected area and spraying said affected area with a composition comprising an ameliorating agent.

In a further embodiment, this invention relates to a method of treating a vaginal condition comprising cleaning an affected area, spraying said affected area with a composition comprising an ameliorating agent and applying a cooling agent.

In another embodiment, this invention relates to a method of treating a vaginal condition comprising cleaning an affected area with a wet substrate, inserting an antifungal active agent into the vagina and spraying the affected area with a composition comprising an ameliorating agent.

In another embodiment, this invention relates to a method of treating a vaginal condition comprising cleaning an affected area with a wet substrate, spraying the affected area with a composition comprising an ameliorating agent, and applying a semisolid composition comprising an active antifungal ingredient to the perineum.

In another embodiment, this invention relates to a method of treating a vaginal condition comprising cleaning an affected area with a wet substrate, inserting an antifungal active agent into the vagina, spraying the affected area with a composition comprising an ameliorating agent, and applying a semisolid composition comprising an active antifungal ingredient to the perineum.

### BRIEF DESCRIPTION OF THE FIGURES

FIG. 1 is a plan view of an embodiment of a bottle useful in the methods and kits of this invention.

FIG. 2 is a cross-sectional view of an embodiment of a spray pump that is useful in the methods and kits of this invention.

### DETAILED DESCRIPTION OF THE PREFERRED EMBODIMENTS

One of the objects of this invention is to provide methods and kits for providing rapid relief to the user and treating the groin area including irritated perineal tissues and/or relieving the symptoms of vaginal conditions including infections of the perineum that may require treatment. The methods and kits of this invention utilize ameliorating agents or ingredients to provide relief to itching, burning and pain incident to groin conditions including infections or specifically perineal conditions. The methods and kits may include active agents for treating such conditions related to bacterial, viral or fungal infections, wound management following surgery (e.g., in the perineum), douching, sexual relations, menopause, tight clothing, vulvovaginal conditions that cause pain and discomfort, such as vulvar vestibulitis, interstitial cystitis, vulvar vaginitis or vulvar dynea (vulvodynia) or other medical conditions that result in irritated tissue and that might be cured by application of topical active ingredients.

Thus, in accordance with this invention, compositions appropriate for the affected vaginal area may be used for treatment of burning, pain and discomfort associated with groin conditions in the form of a spray.

As used herein, the term “ameliorating agent” may include local anesthetics or compounds that abolishes the sensation of pain and thereby provides relief. Examples of ameliorating agents include local anesthetics, antihistamines, anti-inflammatories, skin protectants and those ingredients that provide a cooling sensation. The ameliorating agents that may useful in the compositions of this invention may be combined with active agents as set forth below that are capable of treating an infectious or dermatological condition.

Local anesthetics and antihistamines that are useful in the compositions of this invention include pramoxine, benzocaine, lidocaine, dibucaine, benzyl alcohol, camphor, resorcinol, menthol and diphenhydramine hydrochloride and the like.

Anti-inflammatories such as corticosteroids, including hydrocortisone acetate, may also be employed in the external topical compositions of this invention. COX 2 Inhibitors may also be used, such as Valdecoxib, Celocoxib and Refecoxib. Non-steroidal anti-inflammatory drugs (NSAIDS) such as Indomethacin, Naproxen Sodium, Naproxen Potassium, Diclofenac sodium, Oxaproxin, Salicylate, Etodolac, Meloxicam, Ketoprofen, Tolmecytin sodium, Choline Magnesium and Trisalicylate may also be useful in the compositions and devices of this invention. External topical compositions of this invention may also contain skin protectants. By protecting the skin, not only does the composition soothe the site of infection; it also maintains the integrity of the skin to prevent additional damage and pain. Skin protectants may include allantoin, cocoa butter, dimethicone, kaolin, shark liver oil, petrolatum, vegetable oils, zinc oxide and others known to those of skill in the art.

Other beneficial ingredients or other chemical agents may also be included in order to convey to the patient a sensation of cooling. Sensations such as cooling may provide the perception of relief to the user, especially if the inflamed area is infected. These beneficial ingredients include lower alcohols, menthol, camphor, sorbitol, sugars such as monosaccharides, disaccharides, oligosaccharides, polysaccharides, plant extracts such as aloe, witch hazel, chamomile, hydrogenated soy oil and colloidal oatmeal, and vitamins such as vitamins A, D or E or the like may also be included.

As used herein, the term “active agent” shall include those ingredients such as those having antimicrobial activity including antifungal properties.

In one embodiment, the composition of this invention includes an active agent such as an antimicrobial. Such antimicrobials includes, but are not limited to, antifungals, antibacterials, antivirals, antibiotics, and the like. Antifungal ingredients may include azoles, more preferably imidazoles and more specifically, miconazole nitrate, clotrimazole, econazole, albaconazole, ravuconazole, saperconazole, terconazole, ketoconazole, butaconazole, tioconazole, fluconazole, secnidazole, metronidazole, vericonazole, fenticonazole, sertaconazole, posaconazole, bifonazole, oxiconazole, sulconazole, elubiol, vorconazole, isoconazole, flutrimazole and their pharmaceutically acceptable salts and the like. Other antifungal agents may include an allylamine or one from other chemical families, including but not limited to, ternafine, naftifine, amorolfine, butenafine, ciclopirox, griseofulvin, undecyclenic acid, haloprogin, tolnaftate, nystatin, iodine, rilopirox, BAY 108888, purpuromycin and their pharmaceutically acceptable salts.

In another embodiment of the invention, the spray includes compositions for vulvovaginal use containing one or more antibacterials. The antibacterial may be chosen from the group including, but not limited to, metronidazole, clindamycin, tinidazole, omidazole, secnidazole, refaximin, trospectomycin, purpuromycin and their pharmaceutically acceptable salts and the like.

Antiviral active ingredients include Acyclovir, emtricitabine, ribavirin, adefovir, dipivioxil, tenefovir, retrovir, epivir, indinavir, lamivudin, emetricitabine, sequnavir, hydroxyurea, fosamprenavir and the like.

Another embodiment of the compositions of this invention include compositions for vulvovaginal use containing one or more antiviral agents. Antiviral agents may also include, but are not limited to, immunomodulators, more preferably imiquimod, its derivatives, podofilox, podophyllin, interferon alpha, reticolos, cidofovir, nonoxynol-9 and their pharmaceutically acceptable salts and the like.

Compositions that may be useful in accordance with this invention may contain from about 5 to about 20% w/w of a lower alkyl alcohol. More preferably, they should contain from about 5 to about 15% w/w of a lower alkyl alcohol. Most preferably, it should contain about 10 to about 15% w/w of a lower alkyl alcohol. They may contain from about 0 to about 5% w/w of a solubilizing agent, such as polysorbate 20.

May contain antioxidants, chelating agents, stabilizing agents provided that the composition may be sprayed.

The ameliorating agent is delivered in a pharmaceutically acceptable carrier, such as water, glycerin, mineral oil, a lower alkyl alcohol or the like, known to those of skill in the art, which in its deliverable form is capable of being sprayed. Preferred embodiments include those in which the compositions of this invention are in the form of a spray that includes water and ethyl alcohol, an ameliorating agent such as pramoxine HCl and additional soothing components, such as tocopheryl acetate and/or aloe extract. This spray composition may also contain additional ameliorating agents. For example, the spray composition may also contain a local anesthetic such as benzocaine, lidocaine, dibucaine, tetracaine, diphenyhydramine hydrochloride, tripelennamine, hydrochloride, praxomoxine hydrochloride, butamben picrate and resorcinol. Spray compositions of this invention may further contain anti-inflammatory compounds such as hydrocortisone and others known to the art.

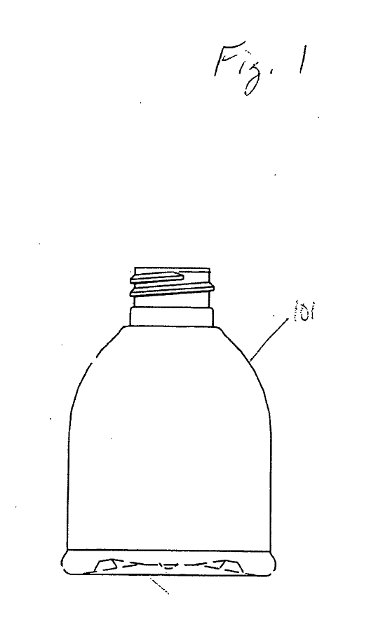

A spray bottle useful in the kits of this invention is illustrated in FIG. 1. Walls 101 are rounded so as to fit easily in the hand of the user. Floor 102 is flat so as to enable easy storage.

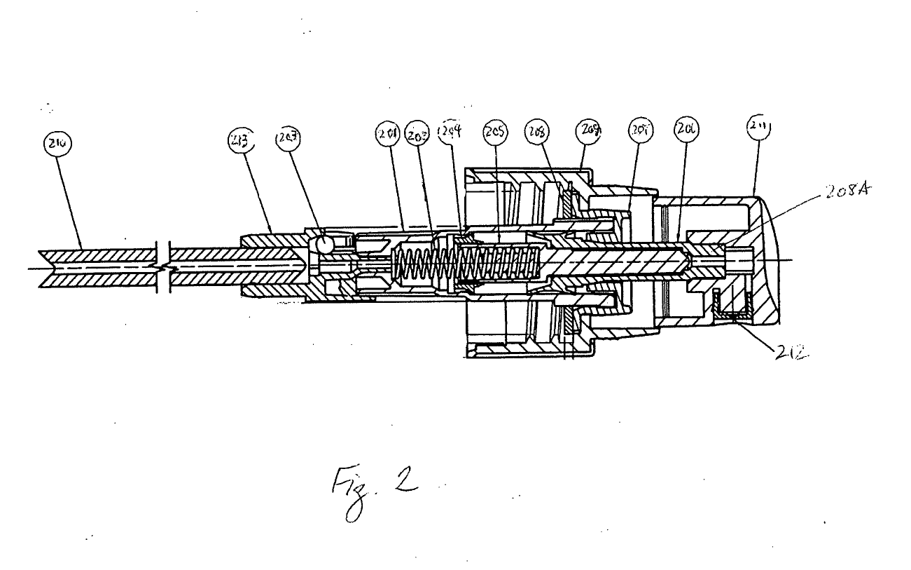

FIG. 2 illustrates a pump that may be useful in the kits of this invention. Housing 201 circumscribes a spring 202. Ball 203 acts as a valve separating a reservoir of composition containing active ingredient from the chamber of the pump from which composition is sprayed. Sliding seal 204 surrounds stem 205, which, in turn, surrounds spring 202. Stem 205 is interconnected with piston 206, which is slidably connected with housing cap 207. Gasket 208 prevents composition from backing into the reservoir once it flows into the dispensing chamber 208A. Screwcap 209 is screwed onto the assembly in order to attach the spray pump to the reservoir. Dip tube 210 is to be placed within the reservoir and is the area through which composition will flow into the spray pump head. Actuator 211 engages with piston 206, which, when depressed toward the reservoir, in turn pushes spring 202, which causes the assembly to be displaced and permits fluid composition to flow into the dispensing chamber through two-way valve 213. Insert 212 protects the fluid from flowing out of the assembly until removed.

The inverted spray bottle of this invention enables the user to apply the spray composition to areas that are difficult or uncomfortable to reach with a bottle that must be used vertical to the ground. It also assists in directing the spray composition to the target area, which can be difficult with conventional spray bottles.

The methods and kits of this invention may also be used to apply treatment compositions for application of hormone-containing products, more particularly estrogen, progestin, or androgen or like active ingredients. Such ingredients and the like may be useful in treating female sexual dysfunction, atrophy due to menopause or other conditions which can be improved by topical application of hormones. In connection with the use of hormones or other dose-sensitive ingredients, the pump or applicator portion of the kit of this invention should be capable of administering a metered dose. Such pumps or applicators are known to those of skill in the art.

The spray compositions of this invention are preferably applied using a reservoir, such as a bottle, and a means for remotely delivering the active ingredient and formulation thereof to the groin area such that the user's hands do not need to contact the groin area. Such a means may be a spray pump, an aerosol pump, or the like. The spray bottle should be in a shape that is comfortable for the user to hold in his or her hand. If a spray pump is used, it should be capable of spraying not only in the upright position, but in other orientations and attitudes, including “upside-down”, or 180° displaced from the upright position.

In one embodiment of the invention, a patient suffering from a vaginal condition, for example, may first clean the vaginal/vulvar area by applying a wipe to remove vaginal discharge. After cleaning the area, the patient picks up and inverts the bottle 180 degrees while maintaining a firm grasp on walls 101. The patient then depresses pump 103, aiming the spray toward the affected area of the perimeum.

In another embodiment, a male suffering from “jock itch”, for example, after showering, may pick up and invert the bottle 180 degrees while maintaining a firm grasp on walls 101. The male then depresses pump 103, aiming the spray toward the affected area of the groin.

In accordance with another embodiment of the methods of this invention, a patient suffering from a vaginal infection, for example, may first clean the vaginal/vulvar area by applying a wipe to remove vaginal discharge. She may then administer her anti-infective medication, either by insertion into the vagina or orally (although if she is treating the infection systemically, she may, optionally, administer the systemic medication prior to local treatment or cleansing). After cleaning the area, the patient should apply an ameliorating composition according to this invention in order to provide relief to the area and may simultaneously apply any local topical treatment that can offer anti-infective and/or analgesic properties to the area.

In another embodiment, the patient additionally after cleaning and spraying the ameliorating composition to the perineum, may also apply a semisolid composition containing an active antifungal ingredient to the perineum. The semisolid composition may preferably be in the form of a cream. More preferably, the antifungal active ingredient utilized in the semisolid composition is miconazole nitrate.

Another method may additionally have the step of applying a semisolid composition containing an active antifungal ingredient to the perineum after cleansing and inserting a dosage form containing an antifungal active ingredient into the vagina. The semisolid composition may preferably be in the form of a cream. More preferably, the antifungal active ingredient utilized in the semisolid composition as well as the dosage form is miconazole nitrate. Most preferably, the dosage form containing miconazole nitrate is contained in a soft gelatin dosage form.

The following examples serve to illustrate, but not limit, the scope of the inventions described herein.

### EXAMPLE 1

Composition 1. (Liquid Spray with Topical Anesthetic)

Pramoxine HCl 1.00%

Ethyl Alcohol 12.00%

Vitamin E 0.005%

Skin conditioning agent 0.8%

Solubilizing agent 0.5%

Purified water 85%

Alcohol is charged to a main tank and mixing is started. The solubilizing agent and Vitamin E are preblended in a separate tank and then added to the alcohol. Water, skin conditioning agent and pramoxine, a local anesthetic, are then added to the main tank and then mixed until the composition is homogenous.

### EXAMPLE 2

Composition 2. (Liquid Spray with Anti-inflammtory Agent)

| | |
|---|---|
| Chelating Agent | 0.01 |
| alcohol | 45 |
| hydrocortisone | 1 |
| glycerin | 20 |
| Surfactant | 0.5 |
| Solubilizing agent | 0.05 |
| pH adjuster | 0.01 |
| water | 33.43 |

Water is charged to a main tank and mixing is started. The chelating agent is then added to the main tank. Hydrocortisone, an anti-inflammatory agent, is educted into the main tank with alcohol and mixed until clear. Glycerin is added and the composition mixed until clear. A premixture of water and surfactant is then added to the main tank. Finally, solubilizing agent and pH adjuster are added and the composition mixed until homogenous.

### EXAMPLE 3

Composition 3. (Liquid Spray with Antihistamine)

Composition 3. (Liquid Spray with Antihistamine)

| | |
|---|---|
| Diphenhydramine HCL | 2 |
| Zinc acetate | 0.1 |
| Alcohol | 12 |
| Glycerin | 2 |
| Povidone | 1 |
| Water | 82.9 |

Water is charged to a main tank and mixing is started. Zinc acetate is then added to the main tank. Diphenhydramine HCL, an antihistamine, is then added to the main tank with alcohol and the composition mixed until clear. Glycerin is added and the composition mixed until clear. A premixture of water and povidone is then added to the main tank and the composition mixed until homogenous.

### EXAMPLE 4

Composition 4.(Liquid Spray with Skin Protectant)

| Phase A | |
|---|---|
| PEG-6 hydrogenated Palamide | 2 |
| Methoxy PEG-22/Dodecyl Glycol Copolymer | 2 |
| Mineral Oil | 10 |
| Dimethicone | 10 |
| Phase B | |
| Sorbitol | 5 |
| Water | 70 |
| preservative | 1 |

Phase A is made by mixing PEG-6 hydrogenated Palamide, Methoxy PEG-22/Dodecyl Glycol Copolymer, mineral oil and dimethicone in a tank until homogenous. Phase B is made by separately mixing sorbitol, water and a preservative together and mixing until homogenous and then added to Phase A and the composition mixed until homogenous.

What is claimed is:

1. A method of treating a vaginal condition comprising spraying a perineum with a composition comprising an ameliorating agent.

2. A method of claim 1, wherein the perineum is sprayed by an inverted pump spray bottle.

3. A method of claim 1, wherein the perineum is sprayed by an inverted aerosol spray bottle.

4. A method of claim 1, wherein the ameliorating agent comprises a local anesthetic.

5. A method of claim 1, wherein the ameliorating agent comprises an antihistamine.

6. A method of claim 1, further comprising a pharmaceutically acceptable carrier.

7. A method of claim 1, wherein the spraying step comprises applying the ameliorating agent from a spray pump.

8. A method of claim 1, wherein the spraying step comprises applying the ameliorating agent from an aerosol container.

9. A method of claim 1, wherein said spraying step comprises applying a composition comprising said ameliorating agent, and a pharmaceutically acceptable carrier to the infected area from a spray pump.

10. A method of claim 1, further comprising an active agent.

11. A method of claim 1, further comprising a cooling ingredient.

12. A method of claim 11, wherein the cooling ingredient comprises from about 5 to about 20% w/w of a lower alkyl alcohol.

13. A method of claim 11, wherein the cooling ingredient comprises from about 5 to about 35% w/w of a polyol.

14. A method of claim 1, further comprising an antifungal compound.

15. A method of claim 14, wherein the antifungal compound is selected from the group consisting of: miconazole nitrate, clotrimazole, econazole, ,albaconazole, ravuconazole, saperconazole, terconazole, ketoconazole, butaconazole, butaconazole, tioconazole, fluconazole, secnidazole, metronidazole, vericonazole, fenticonazole, sertaconazole, posaconazole, bifonazole, oxiconazole, sulconazole, elubiol, vorconazole, isoconazole, flutrimazole and their pharmaceutically acceptable salts, ternafine, naftifine, amorolfine, butenafine, ciclopirox, griseofulvin, undecyclenic acid, haloprogin, tolnaftate, nystatin, iodine, rilopirox, BAY 108888, purpuromycin and their pharmaceutically acceptable salts.

16. A method of claim 10, wherein the active agent is an antifungal.

17. A method of claim 10, wherein the active agent is an antibiotic.

18. A method of claim 17, wherein the antibiotic is selected from the group consisting of: metronidazole, clindamycin, timidazole, ornidazole, secnidazole, refaximin, trospectomycin, purpuromycin and their pharmaceutically acceptable salts.

19. A method of claim 1, further comprising an antibiotic.

20. A method of claim 10, wherein the active agent is an antiviral compound.

21. A method of claim 20, wherein said antiviral compound is selected from the group consisting of: Acyclovir, emtricitabine, ribavirin, adefovir, dipivioxil, tenefovir, retrovir, epivir, indinavir, lamivudin, emetricitabine, sequnavir, hydroxyurea and fosamprenavir.

22. A method of claim 1, further comprising an antiviral compound.

23. A method of claim 1, further comprising a cooling agent.

24. A method of claim 23, wherein the cooling compound comprises lower alcohols, menthol, camphor and sugars.

25. A method of claim 24, wherein said sugar is selected from the group consisting of monosaccharides, disaccharides, oligosaccharides or polysaccharides.

26. A method of claim 24, wherein said sugar is sorbitol.

27. A method of treating a vaginal condition comprising spraying a perineum with a composition comprising at least one ameliorating agent with an inverted spray bottle.

28. A method of treating a vaginal condition comprising spraying a perineum with a composition comprising at least one ameliorating agent.

29. A method of claim 28, wherein the perineum is sprayed by an inverted pump spray bottle.

30. A method of claim 28, wherein the perineum is sprayed by an inverted aerosol spray bottle.

31. A kit for treating a perineal condition comprising an inverted spray bottle and a composition comprising at least one ameliorating agent.

32. A method of treating a groin condition comprising spraying a groin area with a composition comprising an ameliorating agent.

33. A method of claim 32, wherein the groin is sprayed by an inverted pump spray bottle.

34. A method of claim 32, wherein the groin is sprayed by an inverted aerosol spray bottle.

35. A method of treating a vaginal condition comprising cleaning an affected area and spraying said affected area with a composition comprising an ameliorating agent.

36. A method of treating a vaginal condition comprising cleaning an affected area, spraying said affected area with a composition comprising an ameliorating agent and applying a cooling agent.

37. A method of treating a vaginal condition comprising cleaning an affected area with a wet substrate, inserting an antifungal active agent into the vagina and spraying the affected area with a composition comprising an ameliorating agent.

38. A method of treating a vaginal condition comprising cleaning an affected area with a wet substrate, spraying the affected area with a composition comprising an ameliorating agent, and applying a semisolid composition comprising an active antifungal ingredient to the perineum.

39. A method of treating a vaginal condition comprising cleaning an affected area with a wet substrate, inserting an antifungal active agent into the vagina, spraying the affected area with a composition comprising an ameliorating agent, and applying a semisolid composition comprising an active antifungal ingredient to the perineum.

40. A method of claim 40, wherein said semisolid composition is a cream.
